(19) United States
(12) Patent Application Publication
(10) Pub. No.: US 2026/0014154 A1
(43) Pub. Date: Jan. 15, 2026

(54) CYCLIC UREA THIAZOLYL COMPOUND FOR TREATMENT OF VIRAL INFECTIONS UNRESPONSIVE TO TREATMENT WITH A FIRST ANTIVIRAL THERAPY

(71) Applicant: Assembly Biosciences, Inc., South San Francisco, CA (US)

(72) Inventor: Ran YAN, South San Francisco, CA (US)

(21) Appl. No.: 19/266,094

(22) Filed: Jul. 10, 2025

Related U.S. Application Data
(60) Provisional application No. 63/670,717, filed on Jul. 12, 2024.

(51) Int. Cl. A61K 31/513; A61K 31/4439; A61K 31/522; A61P 31/22

(52) U.S. Cl. CPC A61K 31/513; A61K 31/4439; A61K 31/522; A61P 31/22

(57) ABSTRACT

The present disclosure provides methods and pharmaceutical formulations for the treatment and prophylaxis of a viral infection unresponsive to treatment with one or more first antiviral therapy using a cyclic urea thiozole compound.

### CROSS REFERENCE TO RELATED APPLICATION

This application claims the benefit of U.S. Provisional Application No. 63/670,717, filed Jul. 12, 2024, which is incorporated herein by reference in its entirety.

### BACKGROUND

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (i.e., α, β and γ) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and human herpes viruses 6-8 (HHV 6-8).

HSV1 and 2 infections may cause disease in immune competent individuals. Both subtypes cause cutaneous genital/anal and orolabial/nasal cavity (cold sore) lesions, although HSV2 is more commonly associated with the former and HSV1 the latter such that >80% of genital infections are believed to be caused by HSV2. Globally, over 500 million people have genital herpes infections. Symptoms vary but are typically most severe on first time of infection and may last for weeks to months. Approximately 50 to 80% of the world's population have orolabial HSV infection, which is the main cause of cold sores. HSV, and particularly HSV1, may also cause lesions on the fingers (Whitlows) and other areas of the skin.

The vast majority of HSV infected individuals will not experience any noticeable symptoms. However, some will experience recurrent outbreaks of infection. In the USA, 20 to 40% of the population will get recurrent labial HSV lesions. Significantly, orolabial cold sores and Whitlow's provide a very easy route for transmission of the virus to other individuals which may lead to rarer but much more serious HSV-related pathologies. For example, HSV-related ocular keratitis is a major cause of blindness. HSV may also cause encephalitis in neonates which is a life-threatening condition. Other disorders also believed to be caused by HSV include herpes gladiatorum, Mollaret's meningitis and possibly Bell's palsy.

Primary infection with, or reactivation of an existing herpes virus infection, may be a major cause of disease in immunocompromised individuals. Key at-risk immunocompromised populations include patients undergoing solid organ or stem cell transplantation, individuals with HIV/AIDS, and ICU patients.

Presently, there is no cure for HSV. Medicines have been developed that may to some degree prevent or shorten outbreaks, but there is a need for improved therapies for treating HSV infection and inhibiting viral replication.

Currently, nucleoside analogues, such as acyclovir and its prodrugs, e.g., valacyclovir and famciclovir, are used as agents against herpes viruses such as HSV. In order to exert their effects, these nucleoside analogues must first be phosphorylated by viral thymidine kinase (TK) and then subsequently converted by cellular kinases to the nucleoside triphosphate, which inhibits the activity of the viral DNA polymerase. If the virus has no functionally active TK, as is the case, for example, with resistant HSV mutants or with TK-negative viruses, the active substance is unable to exert its effects.

Nucleoside analogues are clinically administered at a dose as high as several hundred in mg to several grams per day and even in high doses, and over long treatment durations, these compounds do not completely prevent recurrent outbreaks of symptoms from HSV infection. High doses also lead to increased levels of adverse effects.

Viral shedding is also common in HSV patients and may asymptomatically facilitate the transmission of HSV to more individuals. Nucleoside analogues do little to address this and long-term suppressive treatment, e.g., with valacyclovir has been shown to reduce transmission risk only by 46%. Since the nucleoside analogues may incorporate into the genome DNA of a host via the host DNA polymerase, the mutagenicity of these agents is also a concern, as documented for the nucleoside analogue, ganciclovir.

Given the inadequacy of existing treatments, there is an urgent medical need to develop improved, well-tolerated anti-herpes treatments.

A class of compounds being investigated for HSV treatment are the helicase-primase inhibitors. Helicase-primase inhibitors are antiviral agents with a novel mechanism of action against HSV1 and 2. They inhibit the viral heterotrimeric complex consisting of helicase, primase, and cofactor subunits that have functions essential for viral DNA replication. They are not nucleoside analogues and do not require phosphorylation by TK to inhibit HSV replication and they are therefore potentially active against TK-deficient HSV, which as described above, is a major mechanism of resistance to nucleoside analogues, such as acyclovir.

Two examples of helicase-primase inhibitors are BILS-179 BS and amenamevir (Katsumata et al. (2018) Biochem Pharm 158 p201-206). BILS-179 BS has been dosed orally but was suspended from early clinical trials due to adverse events. Another example is pritelivir, a thiazolylamide derivative with the chemical name N-[5-(aminosulfonyl)-4-methyl-1,3-thiazol-2-yl]-N-methyl-2-[4-(2-pyridinyl)-phenyl]acetamide.

WO2001047904 discloses thiazolyl amide derivatives and their use as antiviral medicaments. WO2000053591 discloses thiazolyl derivatives and their utilization as antiviral agents. WO2017174640 discloses aminothiazole derivatives useful as antiviral agents. WO2019068817 discloses enantiomers of substituted thiazoles as antiviral compounds. WO2024049760 discloses cyclic urea thiazolyl compounds, and pharmaceutical compositions thereof, and methods of the treatment and prophylaxis of HSV infections.

There is still a need for additional antiviral compounds for the treatment and prophylaxis of HSV infections that are unresponsive to treatment with one or more first antiviral treatments and have an improved profile with respect to safety, potency, selectivity and/or bioavailability.

### SUMMARY OF THE INVENTION

In one embodiment described herein is a method for the treatment of a viral infection unresponsive to treatment with one or more first antiviral therapy, in a subject in need thereof, the method comprising: administering to the subject a therapeutically effective amount of Compound 1:

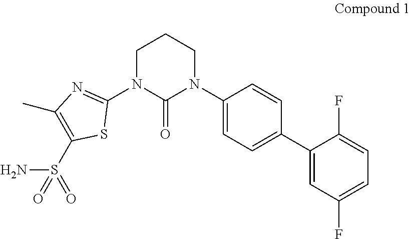 [SMILES: Cc1nc(N2CCCN(c3ccc(-c4cc(F)ccc4F)cc3)C2=O)sc1S(N)(=O)=O]

or a pharmaceutically acceptable salt thereof.

In one aspect of the method, first antiviral therapy is a nucleoside analogue or a helicase-primase inhibitor. In one aspect of the method, nucleoside analog is acyclovir. In one aspect of the method, the helicase-primase inhibitor is pritelivir. In one aspect of the method, the infection is a Herpes Simplex Virus (HSV) infection. In one aspect of the method, the HSV is an HSV-1 or HSV-2. In one aspect of the method, the HSV comprises one or more mutations. In one aspect of the method, the HSV comprises a T288M mutation in the UL23 gene. In one aspect of the method, the infection is resistant to treatment with one or more antiviral therapy.

In one aspect of the method, Compound 1 is about 400 fold more potent than acyclovir. In one aspect of the method, Compound 1 is about 4 fold more potent than pritelivir.

Another embodiment described here in a method for the treatment of a viral infection unresponsive to treatment with one or more first antiviral therapies in a subject in need thereof, the method comprising: administering to the subject a therapeutically effective amount of a pharmaceutical composition comprising Compound 1:

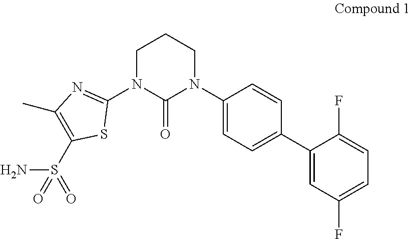 [SMILES: Cc1nc(N2CCCN(c3ccc(-c4cc(F)ccc4F)cc3)C2=O)sc1S(N)(=O)=O]

or a pharmaceutically acceptable salt thereof and a pharmaceutically acceptable excipient. In one aspect of the method, antiviral therapy is a nucleoside analogue or a helicase-primase inhibitor. In one aspect of the method, nucleoside analogue is acyclovir. In one aspect of the method, helicase-primase inhibitor is pritelivir. In one aspect of the method, infection is an HSV infection. In one aspect of the method, the HSV is an HSV-1 or HSV-2. In one aspect of the method, HSV is comprises one or more mutation. In one aspect of the method, the HSV comprises a T288M mutation in the UL23 gene. In one aspect of the method, the infection is resistant to treatment with one or more antiviral therapies.

In one aspect of the method, Compound 1 is about 400 fold more potent than acyclovir. In one aspect of the method, Compound 1 is about 4 fold more potent than pritelivir.

Another embodiment described herein is a method for the treatment of HSV infection previously treated with acyclovir, in a subject in need thereof, the method comprising: administering to the subject a therapeutically effective amount of Compound 1:

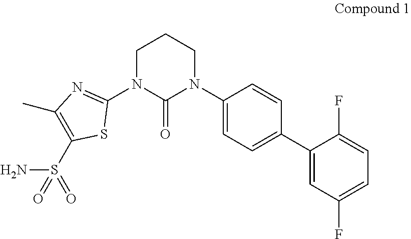 [SMILES: Cc1nc(N2CCCN(c3ccc(-c4cc(F)ccc4F)cc3)C2=O)sc1S(N)(=O)=O]

or a pharmaceutically acceptable salt thereof.

Another embodiment described herein is a method for the treatment of HSV infection previously treated with pritelivir, in a subject in need thereof, the method comprising: administering to the subject a therapeutically effective amount of Compound 1:

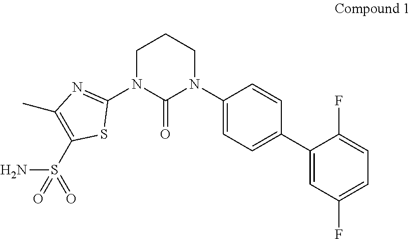 [SMILES: Cc1nc(N2CCCN(c3ccc(-c4cc(F)ccc4F)cc3)C2=O)sc1S(N)(=O)=O]

or a pharmaceutically acceptable salt thereof.

Another embodiment described herein is a method of treatment of an HSV infection previously treated with an antiviral therapy in a subject in need thereof, the method comprising: administering to the subject a therapeutically effective amount of Compound 1:

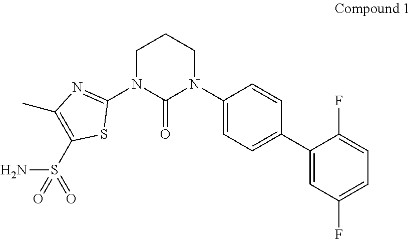 [SMILES: Cc1nc(N2CCCN(c3ccc(-c4cc(F)ccc4F)cc3)C2=O)sc1S(N)(=O)=O]

or a pharmaceutically acceptable salt thereof; wherein the HSV comprises one or more mutation.

In one aspect, the HSV comprises a T288M mutation in the UL23 gene.

### BRIEF DESCRIPTION OF THE DRAWINGS

FIG. 1 describes data demonstrating the broad activity of Compound 1 against HSV-1 and HSV-2 clinical isolates.

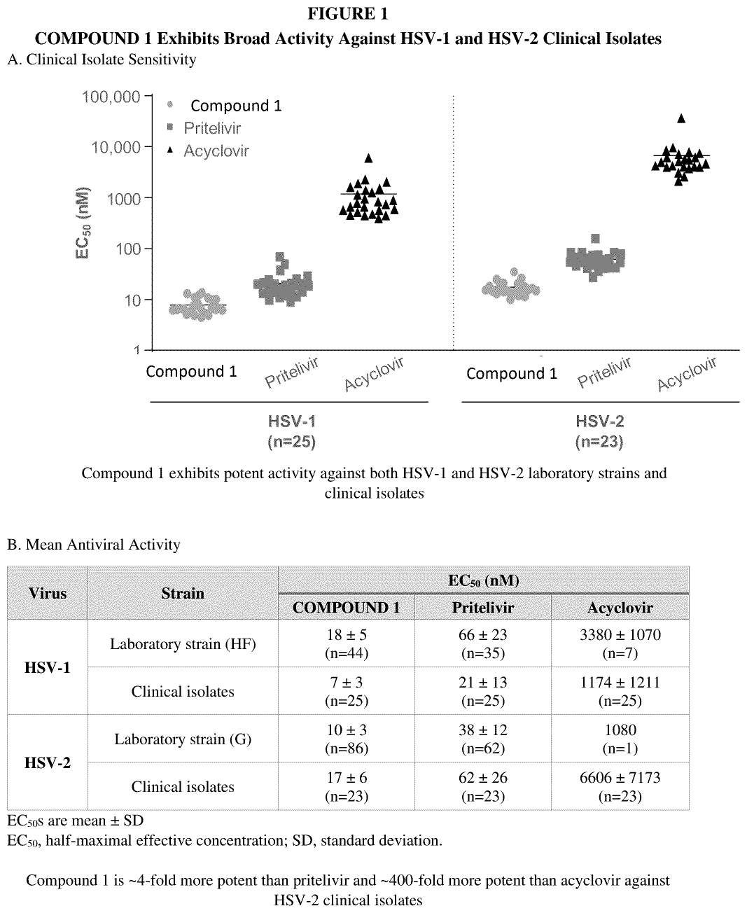

FIG. 2 describes data demonstrating the ability of Compound 1 to target the HP complex.

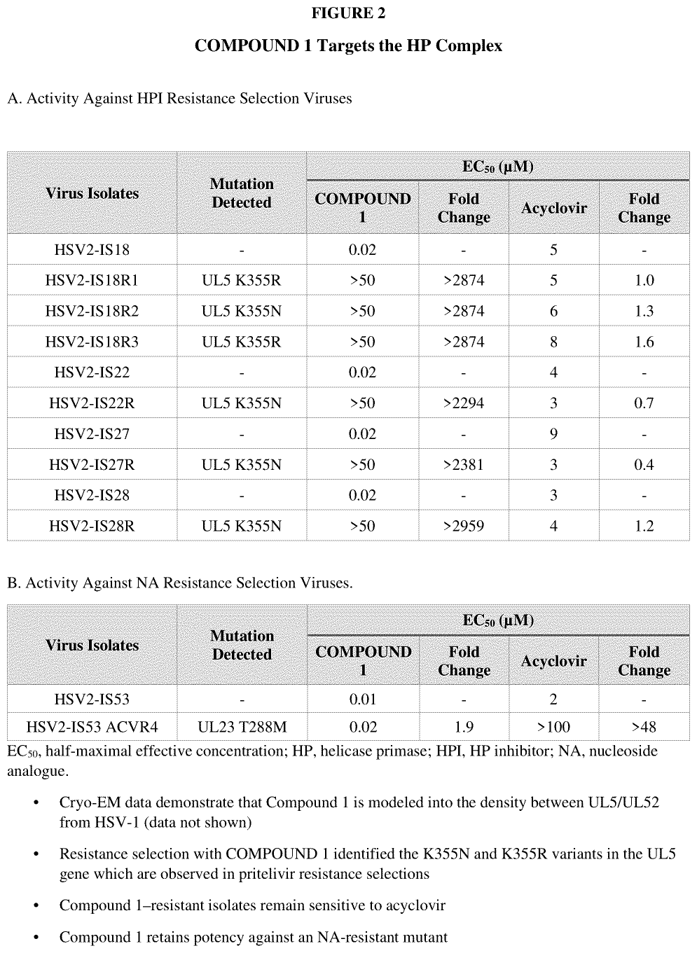

FIG. 3 describes the combination studies with Compound 1 and acyclovir.

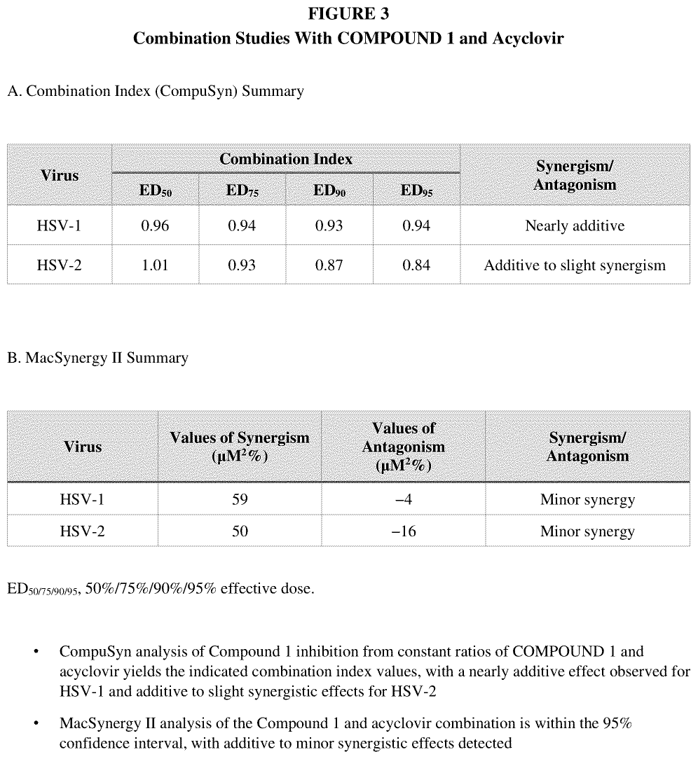

FIG. 4 describes the activity of Compound I specific to HSV and lack of cytotoxicity.

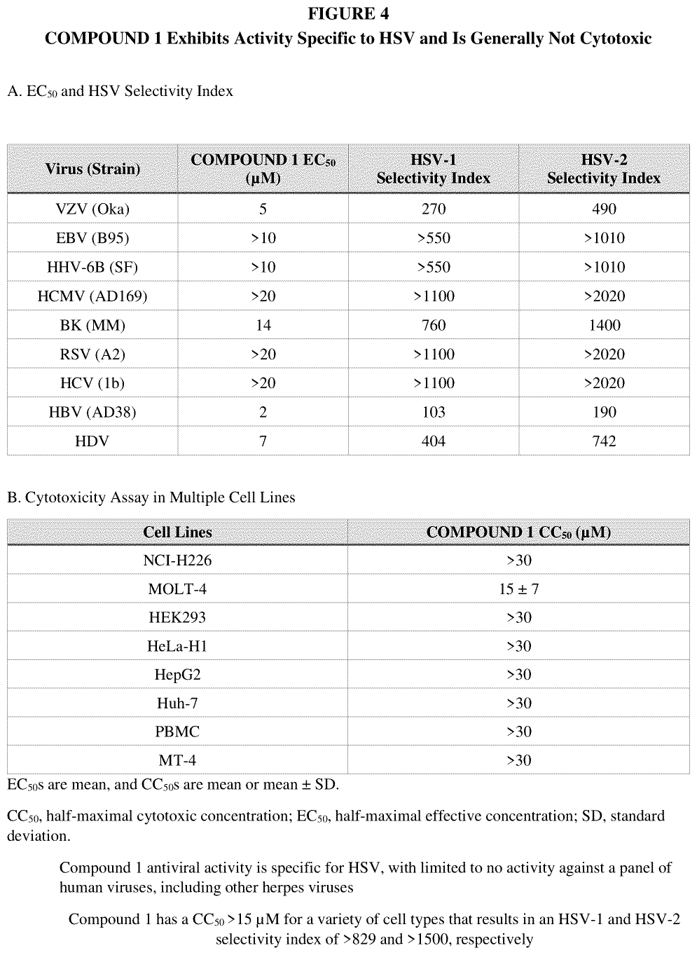

FIG. 5 describes that no off-target effects of Compound 1 are observed in vitro. Compound 1 inhibits HSV over potential human off-target CAs I, II, and III with a selectivity index of >500. A favorable safety profile of Compound 1 is observed in rats and dogs in 28-day oral toxicity studies, with high safety margins relative to the predicted human equivalent dose (data not shown).

FIG. 6 describes pK profile of Compound 1 after a single dose of 100 mg in dogs (FIG. 6A) and pK profile of Compound 1 after IM dose of 400 mg in dogs (FIG. 6B). In dog PK studies, an oral or injectable dose of Compound 1 results in sustained therapeutic plasma concentrations for approximately 2 weeks and more than 3 months, respectively, demonstrating the long-acting potential of Compound 1.

### DETAILED DESCRIPTION OF THE INVENTION

The present disclosure relates to novel uses of Compound 1:

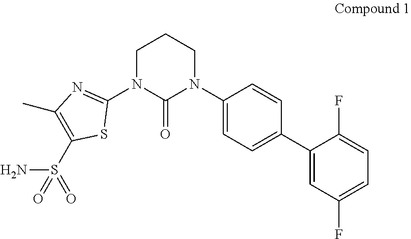 [SMILES: Cc1nc(N2CCCN(c3ccc(-c4cc(F)ccc4F)cc3)C2=O)sc1S(N)(=O)=O]

for the treatment a viral infection unresponsive to treatment with one or more first antiviral therapy. Compound 1 was previously disclosed in WO2024049760, which describes pharmaceutical compositions thereof, and methods for its use in the treatment and prophylaxis of HSV infections.

### Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” can mean one element or more than one element.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B, B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” or “some aspects”, “an aspect” or “one aspect” means that a particular feature, structure, or characteristic described in connection with the embodiment and/or aspect is included in at least some embodiments and/or aspects, but not necessarily all, of the present disclosure.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

As used herein, the term “about” or “approximately” refers to a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 10%, 5%, or 1%.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure may be administered to a mammal, such as a human, but may also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HSV infection is desired.

The term “Pharmaceutically acceptable” includes molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature may form a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-rnethylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound may exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease.

Alternatively, a therapeutically effective amount of a compound is the quantity required to achieve a desired therapeutic and/or prophylactic effect.

The term “treating” includes any effect, e.g., lessening, reducing, modulating, providing a protective effect, or eliminating, a viral infection, that results in the improvement of the disease.

The compounds disclosed herein may exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure, which are identical to those recited herein, except that one or more atoms are replaced by an atom having an atomic mass or mass number different from the atomic mass or mass number usually found in nature. Examples of isotopes that may be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively. For example, a compound of the disclosure may have one or more H atom replaced with deuterium.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure may generally be prepared by following procedures analogous to those disclosed in the examples herein by substituting an isotopically labeled reagent for a non-isotopically labeled reagent.

### Methods of Use:

Thus, one embodiment of the present disclosure provides a method for the treatment of a viral infection unresponsive to treatment with one or more first antiviral therapy in a subject in need thereof, comprising: administering to the subject a therapeutically effective amount of Compound 1:

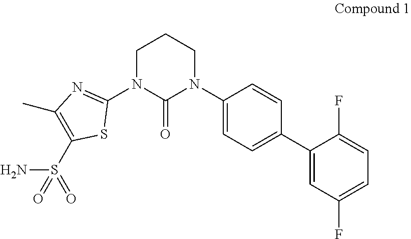 [SMILES: Cc1nc(N2CCCN(c3ccc(-c4cc(F)ccc4F)cc3)C2=O)sc1S(N)(=O)=O]

or a pharmaceutically acceptable salt thereof.

In another aspect, the disclosure provides a method for the treatment of a first viral infection unresponsive to treatment with one or more antiviral therapy in a subject in need thereof, comprising administering to the subject a pharmaceutical composition comprising a therapeutically effective amount of Compound 1, or a pharmaceutically acceptable salt thereof, and a pharmaceutically acceptable excipient.

The term “unresponsive to treatment” as used herein refers to a response from administration of an antiviral therapy, or a combination of therapies, that does not result in an therapeutically effective reduction of viral load, results in no decrease in time of infection without therapeutically effective reduction of viral load, results in a diminished reduction of viral load or reduced potency as compared to the one or more compounds administered and described herein, or does not result in an improvement of symptoms.

An unresponsive response may be due to a variety of underlying mechanisms, including drug resistance. For example, in the case of HSV antiviral therapy, the HSV may become resistant to acyclovir due to mutations in the viral thymidine kinase gene resulting from exposure to the drug. Thus, in one aspect, the infection is resistant to acyclovir. In another aspect, HSV comprises one or more mutations resulting from exposure to acyclovir. In one aspect, the mutation is a T288M mutation in the UL23 gene, however other mutations may also result in resistance to acyclovir and are contemplated herein. In other aspects, HSV may become resistant to a pritelivir due to mutations in the helicase-primer gene. In one aspect, the infection is resistant to pritelivir. In another aspect, the HSV comprises one or more mutations in the UL5 gene. In one aspect, the mutation is a K355R or K355N mutation in the UL5 gene, however other mutations may also result in resistance to pritelivir and are contemplated herein. Thus, in one aspect described herein, the infection may be resistant to treatment with one or more antiviral therapy.

Alternatively, unresponsive to a treatment may also be the result of varying potency. Thus, while a first antiviral therapy, or combination, may have a first potency that is less than a subsequent therapy, which subsequent therapy is considered more potent.

As noted in the Examples, Compound 1 of the present disclosure displays enhanced antiviral activity against HSV as compared to other antiviral therapies. Thus, in one aspect described herein, administration of Compound 1 may result in about a 2 fold, about a 4 fold, about a 10 fold, about a 20 fold, about a 30 fold, about a 40 fold, about a 50 fold, about a 60 fold, about a 70 fold, about a 80 fold, about a 90 fold, a about 100 fold, about a 200 fold, about a 300 fold, about a 400 fold, about a 500 fold, about a 600 fold, about a 700 fold, about a 800 fold, about a 900 fold, about a 1,000 fold, about a 2,000 fold, about a 3,000 fold, about a 4,000 fold, about a 5,000 fold, about a 6,000 fold, about a 7,000 fold, about a 8,000 fold, about a 9,000 fold, or about a 10,000 fold increase in potency of the first antiviral therapies. In one aspect, Compound 1 is about 2 fold to about 10 fold more potent than a first antiviral therapy, inclusive of intermittent values. In another aspect Compound 1 is about 100 fold to about 500 fold more potent than a first antiviral therapy, inclusive of intermittent values.

In one aspect, Compound 1 is about 4 fold more potent than pritelivir. In another aspect, Compound 1 is about 400 fold more potent than acyclovir.

Thus, another embodiment described herein is a method for the treatment of HSV infection having reduced susceptibility to acyclovir, in a subject in need thereof, the method comprising: administering to the subject a therapeutically effective amount of Compound 1, or a pharmaceutically acceptable salt or solvate thereof:

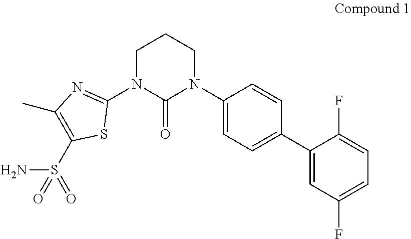 [SMILES: Cc1nc(N2CCCN(c3ccc(-c4cc(F)ccc4F)cc3)C2=O)sc1S(N)(=O)=O]

Another embodiment described herein is a method for the treatment of HSV infection having reduced susceptibility to pritelivir, in a subject in need thereof, the method comprising: administering to the subject a therapeutically effective amount of Compound 1:

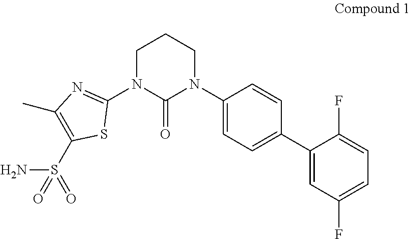 [SMILES: Cc1nc(N2CCCN(c3ccc(-c4cc(F)ccc4F)cc3)C2=O)sc1S(N)(=O)=O]

or a pharmaceutically acceptable salt thereof.

The term “reduced susceptibility” as used herein refers to a reduction in the effect of the administered drug.

Another embodiment described herein is a method of treatment of an HSV infection previously treated with an antiviral therapy in a subject in need thereof, the method comprising: administering to the subject a therapeutically effective amount of Compound 1:

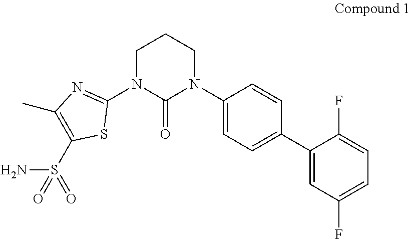 [SMILES: Cc1nc(N2CCCN(c3ccc(-c4cc(F)ccc4F)cc3)C2=O)sc1S(N)(=O)=O]

or a pharmaceutically acceptable salt thereof; wherein the HSV virus is not a wild-type virus or wherein the HSV virus is mutated as a result of exposure to a first antiviral therapy.

The type or class of the first antiviral therapy may vary. In one aspect of the methods described herein, the first antiviral therapy may be any known antiviral therapy or class of drugs used to treat the specific viral infection. In some aspects, the antiviral therapy may be a nucleoside analogue. Exemplary antiviral nucleoside analogues include, but are not limited to, acyclovir and its prodrugs, e.g., valacyclovir and famciclovir, penciclovir, or ganciclovir. In one aspect, the antiviral nucleoside analogue is acyclovir. In other aspects, the antiviral therapy may be a replication inhibitor, or specifically, for example, a helicase-primase inhibitor, including, for example pritelivir or amenamevir. In some aspects, the helicase-primase inhibitor is pritelivir.

In some aspects, the infection may be a viral infection. In some aspects, the virus may be any virus, including but not limited to Respiratory Syncytial virus (RSV), Herpes Simplex virus (HSV), Influenza (Flu), Rhinovirus, Adenovirus, Parainfluenza, Coxacki, or Coronavirus. In one aspect, the infection is an HSV infection. HSV infections may include, but are not limited to, three subfamilies (i.e., α, β and γ) based upon their biological characteristics and the family consists of eight members, i.e., Herpes Simplex Virus subtype type 1 and 2 (HSV-1, HSV-2), Varicella Zoster Virus (VZV), Epstein-Barr virus (EBV), Cytomegalovirus (CMV), and human herpes viruses 6-8 (HHV 6-8). In one aspect, the infection is an HSV-1 infection. In another aspect, the infection is an HSV-2 infection.

In another aspect, the virus may contain one or more mutation. In one aspect, the HSV virus comprises one or more mutations. In another aspect, the HSV comprises one or mutations in the UL23 gene. In another aspect, the HSV virus comprises a T288M mutation in the UL23 gene. In another aspect, the HSV comprises one or more mutations in the UL5 gene. In one aspect, the HSV comprises a 1K355R or K355N mutation in the UL5 gene. Other mutations and combinations of mutations within genes and between genes are contemplated herein.

In some aspects, the subject is a herpes-positive patient. In other aspects, the subject is a herpes-simplex-positive patient. In some aspects, the infection is a Herpes simplex infection and the subject displays symptoms such as Herpes labialis, Herpes genitalis, HSV-related keratitis, encephalitis, or pneumonia. In another aspect, the infection is a Herpes simplex infection and the subject displays symptoms such as suppressed immune system (for example AIDS patients, cancer patients, patients having a genetic immunodeficiency, transplant patients). In another aspect, the infection is a Herpes simplex infection, and the subject is a new-born child or infant.

### Combination Therapies

The compounds according to the present disclosure are also useful for the treatment of a viral infection, in particular where the viral infection is a Herpes simplex viral infection, that is unresponsive to treatment with one or more first antiviral therapy, in combination with other active ingredients.

In one aspect, the present disclosure provides a method for the treatment of a viral infection unresponsive to treatment with one or more first antiviral therapy, and in particularly in a subject in need thereof, the method comprising: administering to the subject a therapeutically effective amount of Compound 1, or a pharmaceutically acceptable salt thereof, in combination with an antiviral therapy, including in combination with the first antiviral therapy, despite the infection being unresponsive to treatment with the first antiviral therapy. Thus one aspect of the present method comprises administering Compound 1, or a pharmaceutically acceptable salt thereof, in combination with the first antiviral therapy.

In some aspects, the antiviral therapy is selected from the group consisting of acyclovir, penciclovir, famciclovir, ganciclovir and valacyclovir, foscarnet trifluridine, pritelivir or amenamevir. In some aspects, the first antiviral therapy is selected from the group consisting of acyclovir, penciclovir, famciclovir, ganciclovir and valacyclovir, foscarnet, trifluridine, pritelivir or amenamevir.

Even when an HSV virus is resistant to either acyclovir or pritelivir alone, administration of either compound in combination with Compound 1 may be effective. Thus, in one aspect, Compound 1 may be administered with acyclovir, where acyclovir is considered the first antiviral therapy. In yet another aspect, Compound 1 may be administered with pritelivir, where pritelivir is considered the first antiviral therapy.

### Formulations and Administration

Compound 1 of the present disclosure may be converted in a known manner into the customary formulations, such as tablets, sugar-coated tablets, pills, granules, aerosols, syrups, emulsions, suspensions, and solutions, using inert, nontoxic, pharmaceutically suitable carriers and solvents. Here, the therapeutically active compound should in each case be present in a concentration of about 0.5 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, it being possible, for example, if the diluent used is water, to use, if appropriate, organic solvents as auxiliary solvents.

Administration is carried out in a customary manner, including orally, parenterally, topically, perlingually or intravenously.

In the case of parenteral administration, solutions or suspensions of the active compounds using suitable liquid carrier and excipients may be employed.

In general, it has proved advantageous in the case of intravenous administration to administer amounts of from approximately 0.001 to 20 mg/kg, preferably approximately 0.01 to 10 mg/kg, of bodyweight to achieve effective results, and in the case of oral administration the dose is approximately 0.01 to 30 mg/kg, preferably 0.1 to 20 mg/kg, of bodyweight.

In some instances, it may be necessary to depart from the amounts mentioned, namely depending on the bodyweight or on the type of administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of the administration of relatively large amounts, it may be advisable to divide this into several individual administrations over the course of the day.

If appropriate, it may be useful to combine the compounds according to the invention with other active substances, in particular antiviral active substances.

The compounds used in the present disclosure may be in the form of a pharmaceutically acceptable salt, cocrystal or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups may be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e., groups which may be protonated, may be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesuifonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts may be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which may be used, for example, as intermediates for chemical reactions or for the preparation of pharmaceutically acceptable salts.

In practical use, Compound 1 may be combined as the active ingredient in intimate admixture with a pharmaceutical carrier according to conventional pharmaceutical compounding techniques. The carrier may take a wide variety of forms depending on the form of preparation desired for administration, e.g., oral, or parenteral {including intravenous). In preparing the compositions for oral dosage form, any of the usual pharmaceutical media may be employed, such as, for example, water, glycols, oils, alcohols, flavouring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds may also be administered intranasally as, for example, liquid drops or spray or as eye drops.

The tablets, pills, capsules, and the like may also contain a binder such as hydroxypropyl methylcellulose, or polyvinylpyrrolidone; diluent or fillers such as microcrystalline cellulose, dicalcium phosphate, lactose, or mannitol; a disintegrating agent such as croscarmellose sodium, polyvinylpyrrolidone, or sodium starch glycolate; a lubricant such as magnesium stearate or sodium stearyl fumarate; a glidant such as silicon dioxide; and a sweetening agent such as sucrose or saccharin. When a dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compound 1 may also be administered parenterally. Solutions or suspensions may be prepared in water suitably mixed with a surfactant such as hydroxypropyl cellulose, sodium lauryl sulfate, or polysorbate. Dispersions may also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous), ocular, pulmonary, nasal, and the like may be employed. Dosage forms include tablets, troches, dispersions, suspensions, solutions, capsules, creams, ointments, aerosols, and the like. Compounds of the present invention may be administered orally or as eye drop. The compounds of the present invention may also be administered orally. The effective dosage of active ingredient employed may vary depending on the particular compound employed, the mode of administration, the condition being treated, and the severity of the condition being treated. Such dosage may be ascertained readily by a person skilled in the art.

Compound 1 may also be present in combination with additional active ingredients, in particular, with one or more active ingredients exhibiting advantageous effects in the treatment of any of the disorders or diseases as described herein. Compound 1 may be present in a composition in combination with at least one further active substance being effective in treating a disease or disorder associated with viral infections (antiviral active compounds), preferably a disease or disorder being associated with viral infections caused by herpes viruses, such as in particular by Herpes simplex viruses (i.e., combination therapy). The at least one further active substance being effective in treating a disease or disorder associated with viral infections (antiviral active compounds) are preferably selected from the group consisting of nucleosidic drugs such as acyclovir, valacyclovir, penciclovir, ganciclovir, famciclovir and trifluridine, as well as compounds such as foscarnet and cidofovir.

Accordingly, the present invention further relates to a pharmaceutical composition comprising Compound 1 and at least one pharmaceutically acceptable carrier and/or excipient and/or at least one further active substance being effective in treating a disease or disorder associated with viral infections (antiviral active compounds).

Compound 1 may be converted in a known manner into customary formulations, such as tablets, caplets, sugar-coated tablets, pills, granules, aerosols, syrups, pharmaceutically suitable carriers, and solvents. Here, the therapeutically active compound should in each case be present in a concentration of about 0.1 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, if being possible, for example, if the diluent used is water, to use, if appropriate, organic solvents as auxiliary solvents.

Administration is carried out in a customary manner, preferably orally, parenterally or topically, in particular perlingually or intravenously.

In the case of parenteral administration, solutions or suspensions of the active compounds using suitable liquid carrier materials may be employed.

In general, it has proved advantageous in the case or intravenous administration to administer amounts of from approx. 0.001 to 20 mg/kg, preferably approx. 0.01 to 10 mg/kg of bodyweight to achieve effective results, and in the case of oral administration the dose is approx. 0.01 to 30 mg/kg, preferably 0.1 to 20 mg/kg of body weight.

Despite this, it may be necessary, if appropriate, to depart from the amounts mentioned, namely depending on the bodyweight or on the type of the administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of administration of relatively large amounts it may be advisable to divide this into several individual administrations over the course of the day.

The features and other details of the disclosure will now be more particularly described. Before further description of the present disclosure, certain terms employed in the specification, examples and appended claims are collected here. These definitions should be read in view of the remainder of the disclosure and as understood by a person of skill in the art. Unless defined otherwise, all technical and scientific terms used herein have the same meaning as commonly understood by a person of ordinary skill in the art.

### Examples

### Preparation of Compound 1

Compound 1 may be prepared in several ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, may be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

### Abbreviations:

AcOH Acetic acid
ACN Acetonitrile
Boc₂O Di-tert-butyl dicarbonate
nBuLi n-Butyllithium
DCM Dichloromethane
DIAD Diisopropyl azodicarboxylate
DIEA Diisopropyl ethylamine
DMF N, N-Dimethylformamide
DMSO Dimethyl sulfoxide
DPPF 1,1′-Bis(diphenylphosphino)ferrocene
EtOAc Ethyl acetate
Et₃N Triethylamine
HATU Hexafluorophosphate Azabenzotriazole Tetramethyl Uronium
h, hr Hour(s)
HPLC High performance liquid chromatography
LCMS Liquid chromatography-mass spectrometry
MeOH Methanol
NMO/NMMO N-Methyl morpholine-N-Oxide
NBS N-Bromosuccinimide
PE Petroleum ether
iPrOH Isopropanol
rt, r.t. Room temperature
SFC Supercritical Fluid Chromatography
TEA Triethylamine
TBAI Tetrabutylammonium iodide
TBAB Tetrabutylammonium bromide
TFA Trifluoroacetic acid
THF Tetrahydrofuran
TLC Thin-layer chromatography
XPhos 2-Dicyclohexylphosphino-2′,4′,6′-triisopropylbiphenyl
   Following LCMS Method have been Used for the Analysis of Final Compounds:

Method A: X-Bridge BEH C-18 (3×50 mm×2.5 mm); Mobile phase: A; 0.025% formic acid in H₂O; B; CH₃CN; Injection volume:2 μL; Flow rate:1.2 mL/min, column temperature: 50° C.; Gradient program: 2% B to 98% B in 2.2 min, held 3 min, at 3.2 min B conc. is held at 2% for 4 min.

Method B: X-select CSH 18 (3×50 mm×2.5 mm); Mobile phase: A; 0.025% formic acid in H₂O; B; CH₃CN; Injection volume:2 μL; Flow rate:1.2 mL/min, column temperature: 50° C.; Gradient program: 0% B to 98% B in 2 min, hold for 3 min, at 3.2 min B conc. is held at 0% for 4 min.

Method C: X-select CSH 18 (3×50 mm×2.5 mm); Mobile phase: A; 0.05% formic acid in H₂O:CH₃CN (95:5); B; 0.05% formic acid in CH₃CN; Injection volume: 2 μL; Flow rate: 1.2 mL/min, column temperature: 50° C.; Gradient program: 0% B to 98% B in 2 min, hold for 3 min, at 3.2 min B conc. is held at 0% for 4 min.

Method D: X-select CSH C18 (3×50 mm×2.5 μm); Mobile phase: A; 2 mM in Ammonium Bicarbonate; B; CH₃CN; Injection volume:2 μL; Flow rate:1.2 mL/min, column temperature: 50° C.; Gradient program: 0% B to 98% B in 2 min, hold for 3 min, at 3.2 min B conc. is held at 0% for 4 min.

Method E: X-select CSH 18 (3×50 mm×2.5 mm); Mobile phase: A; 0.05% formic acid in H₂O; B; CH₃CN; Injection volume: 2p L; Flow rate:1.5 mL/min, column temperature: 50° C.; Gradient program: 0% B to 100% B in 1.5 min, hold 2.2 min, at 2.6 min B conc. is held at 0% for 3 min.

### Synthesis of 2-(3-(2′,5′-difluoro-[1,1′-biphenyl]-4-yl)-2-oxotetrahydropyrimidin-1(2H)-yl)-4-methylthiazole-5-sulfonamide (Compound 1)

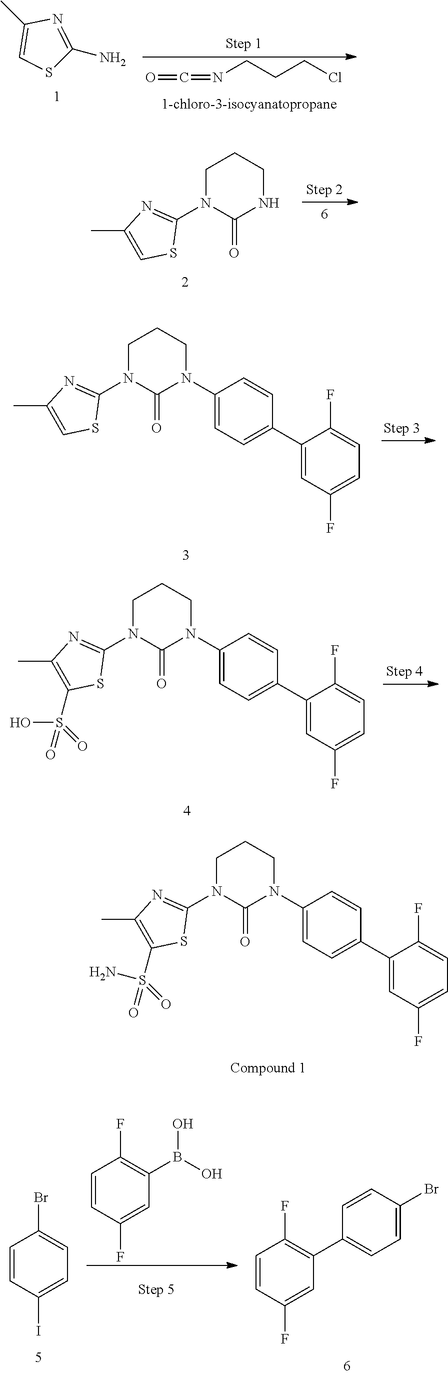

### Step 1. Synthesis of 1-(4-methylthiazol-2-yl) tetrahydropyrimidin-2(1H)-one (compound 2)

A mixture of starting material-compound 1 (6 g, 52.632 mmol) and 1-chloro-3-isocyanatopropane (6.26 g, 52.632 mmol) in THF (60 mL) was heated at 70° C. for 6 h. To the resulting solution, TBAB (1.7 g, 5.263 mmol) and K₂CO₃ (18.15 g, 131.58 mmol) were added portion wise maintaining the same temperature and stirring continued at 70° C. for 16 h. After completion of the reaction (monitored by TLC), the reaction mixture was diluted with water and extracted with EtOAc. The combined organic layers were dried over anhydrous Na₂SO₄, filtered and concentrated under reduced pressure. The crude compound was purified by CombiFlash chromatography (eluting with 60-70% EtOAc in heptane) to afford the title compound 2 (5.1 g, 49.2%) as an off-white solid. TLC: 70% EtOAc/hepatne (R_{f}: 0.5). MS (ESI): calcd. for C₈H₁₁N₃OS: 197.06; Found: 198.17 [M+1]⁺. ¹H NMR (400 MHz, DMSO-d₆): δ 7.30 (s, 1H), 6.60 (s, 1H), 3.99 (t, J=5.4 Hz, 2H), 3.20-3.19 (m, 2H), 2.28 (s, 3H), 1.99-1.89 (m, 2H) ppm.

### Step 2. Synthesis of 1-(2′,5′-difluoro-[1,1′-biphenyl]-4-yl)-3-(4-methylthiazol-2-yl) tetrahydropyrimidin-2(1H)-one (compound 3)

To a stirred solution of compound 2 (5 g, 25.380 mmol) in 1, 4-dioxane (100 mL) were added compound 6 (8.16 g, 30.456 mmol), K₂CO₃ (8.75 g, 63.45 mmol) followed by CuI (0.96 g, 5.076 mmol) and the resulting reaction mixture was purged under nitrogen for 20 min. To this resulting reaction mixture, 1,2-Dimethylethylenediamine (0.9 g, 10.152 mmol) was added under a nitrogen atmosphere. The reaction mixture was heated at 120° C. for 24 h in a sealed tube. The reaction mixture was filtered through Celite bed and washed with ethyl acetate. The filtrate was diluted with water and, extracted with EtOAc then washed with brine. The combined organic layers were dried over anhydrous Na₂SO₄, filtered and concentrated under reduced pressure. The crude compound obtained was purified by CombiFlash chromatography (eluting with 30-40% EtOAc in heptane) to afford the title compound 3 (4.1 g, 41.9%) as an off-white solid. TLC: 50% EtOAc/Heptane (R_{f}: 0.5). MS (ESI): calcd. for C₂₀H₁₁F₂N₃₀S: 385.11; Found: 385.90 [M+1]⁺. ¹H NMR (400 MHz, DMSO-d₆): δ 7.61 (d, J=7.8 Hz, 2H), 7.54-7.35 (m, 4H), 7.35-7.21 (m, 1H), 6.70 (s, 1H), 4.17 (t, J=5.6 Hz, 2H), 3.81 (t, J=4.9 Hz, 2H), 2.26 (s, 3H), 2.24-2.21 (m, 2H) ppm.

### Step 3. Synthesis of 2-(3-(2′,5′-difluoro-[1,1′-biphenyl]-4-yl)-2-oxotetrahydropyrimidin-1(2H)-yl)-4-methylthiazole-5-sulfonic acid (compound 4)

To a stirred solution of compound 3 (4 g, 10.389 mmol) in dry DCM (40 mL) at 0° C. in an inert atmosphere, chlorosulfuric acid (2.07 mL, 31.168 mmol) was added and the resulting reaction mixture was slowly warmed to room temperature and stirred for 12 h. The reaction mixture was concentrated under reduced pressure to dryness. The crude residue obtained was purified by trituration with diethyl ether. The obtained solid was filtered off and dried in vacuo to afford the title compound 4 (3.35 g, crude) as an off-white solid. TLC: 100% EtOAc (R_{f}: 0.2). MS (ESI): calcd. for C₂₀H₁₁F₂N₃O₄S₂: 465.06; Found: 466 [M+1]⁺.

### Step 4. Synthesis of 2-(3-(2′,5′-difluoro-[1,1′-biphenyl]-4-yl)-2-oxotetrahydropyrimidin-1(2H)-yl)-4-methylthiazole-5-sulfonamide (Compound 1)

A stirred solution of compound 4 (3.3 g, 7.096 mmol) in POCl₃ (33 mL) was allowed to stir at 90° C. for 5 h. The reaction mixture was concentrated under reduced pressure to dryness. The resulting residue was dissolved in THF (66 mL), and aqueous ammonia (33 mL) was added at −5° C. while stirring was continued at room temperature for another 12 h. The reaction mixture was diluted with water and extracted with EtOAc. The combined organic layers were dried over anhydrous Na₂SO₄ filtered and concentrated under reduced pressure. The crude compound was purified by CombiFlash chromatography (eluting with 100% EtOAc) to afford the desired product as a white-solid Compound 1 (1.1 g, 44.6%) as a white solid. ¹H NMR (400 MHz, DMSO-d₆): δ 7.65-7.59 (m, 2H), 7.55 (br s, 2H), 7.53-7.48 (m, 2H), 7.48-7.36 (m, 2H), 7.31-7.25 (m, 1H), 4.17 (t, J=6.1 Hz, 2H), 3.82 (t, J=5.6 Hz, 2H), 2.45 (s, 3H), 2.29-2.18 (m, 2H) ppm.

### Step 5. Synthesis of 4′-bromo-2,5-difluoro-1,1′-biphenyl (compound 6)

To a stirred solution of compound 5 (5 g, 17.674 mmol) in 1,4 dioxane: H₂O (50:5 mL) were added (2,5-difluorophenyl) boronic acid (3.07 g, 19.441 mmol) and K₃PO₄ (7.5 g, 35.348 mmol) and the reaction mixture was purged under nitrogen for 10 min. Pd(dppf)Cl₂ (1.29 g, 1.767 mmol) was added under a nitrogen atmosphere. The reaction mixture was heated at 80° C. for 1 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered, and concentrated under reduced pressure. The crude compound was purified by CombiFlash chromatography (eluting with 100% heptane) to afford the title compound 6 (2.3 g, 48.6%) as an off-white solid. TLC: 100% heptane (R_{f}: 0.5). ¹H NMR (400 MHz, CDCl₃): δ 7.58 (d, J=8.3 Hz, 2H), 7.40 (d, J=7.3 Hz, 2H), 7.15-7.06 (m, 2H), 7.05-6.97 (m, 1H) ppm.

### Biological Data

### Cell Culture

Vero, Huh7, HepG2, HEK293, and HeLa-H1A cells were cultured in Dulbecco's Modification of Eagle's Media (DMEM; Cytiva, Cat #SH30243.02) supplemented with 10% heat-inactivated Fetal Bovine Serum (FBS, Corning, Cat #35-010-CV), and 1×Pen/Strep (100 IU/mL penicillin and 100 μg/mL streptomycin; Corning, Cat #30-002-C1). MOLT-4, NCI-H226, and MT-4 cells were cultured in Roswell Park Memorial Institute Media 1640 (RPMI-1640, Cytiva, Cat #12633012) media supplemented with 10% heat-inactivated FBS and 1×Pen/Strep. Hepatitis C virus (HCV)-1b replicon differentiated hepatocytes from hepatocellular carcinoma (Huh7) cells were cultured in DMEM high-glucose containing glutamine and sodium pyruvate (Cytvia, Cat #SH30243.02) supplemented with 10% FBS, 1×Pen-Strep, and 250 μg/mL G418 (Thermo Fisher, Cat #10131035). Freshly frozen PBMCs (Hemacare, Cat #PB009C01, Lot #18046747) were cultured in RPMI-1640 media containing 10% heat-inactivated FBS and 1×Pen/Strep. All cells were cultured at 37° C. with 5% CO₂.

Fetal lung fibroblasts (MRC-5), marmoset B-lymphoblastoid cell line (B95-8), human peripheral blood mononuclear cells (PBMC), and primary human renal proximal tubule epithelial cells (RPTEC/TERT1) were handled according to manufacturer's instructions at WuXi for VZV, EBV, HHV-6, and BK antiviral assays, respectively.

### HSV Strains

Oral and genital swab samples collected from HSV-1 and HSV-2 infected patients were acquired from Discovery Life Sciences (USA). Swab samples were cultured on Vero cells with infection medium for 3-14 days until 100% CPE were observed and then supernatants were collected as clinical isolate stocks.

Clinical isolate stocks were titrated by plaque assay. Serial 10-fold dilutions of clinical isolate stocks were prepared and used to infect Vero cell monolayers in 6-well plates. After a 1-hour incubation, the virus inoculum was aspirated and then the Vero cells were overlayed with 2 mL of maintenance medium containing 1% carboxymethylcellulose (CMC, EMD Millipore, Cat #217277). Plates were incubated for 5 days and then the supernatant was aspirated, cells were fixed with 4% formaldehyde (Sigma-Aldrich, Cat #252549), and stained with crystal violet solution (EMD Millipore, Cat #192-12) to visualize virus plaques. The viral titer for each isolate was determined by counting the average number of plaques per well for each dilution and multiplying by 10 to the power of each dilution to obtain the number of plaque-forming units per milliliter (PFU/mL).

### Example 1—Cytopathic Effect Reduction Assay

Vero cells (ATCC Cat #CCL-881) were grown to 70-90% confluency, washed once with Dulbecco's phosphate buffered saline (DPBS, Corning Cat #21-030-CM), and detached from the culture flask using 0.25% Trypsin-EDTA at 37° C. for 3 minutes. Detached cells were spun at 500 g for 5 min and the cell pellet was resuspended in growth media. Vero cells were seeded onto 96-well flat clear bottom black polystyrene tissue culture-treated microplates (Corning, Cat #3904) in growth media at a density of 8,000 cells per well. The following day, growth media was replaced with 50 μL of infection media (DMEM supplemented with 2% FBS) containing either COMPOUND 1, PTV, or ACV. The compound stock solutions were serially diluted 3-fold for a total of 8 dilutions in 100% DMSO (in triplicate) and were added to each well. All wells were normalized to a final DMSO concentration of 0.5%, 50 μL of either HSV-1 (HF) or HSV-2 (G) virus, diluted 80,000-fold or 1,000-fold, respectively, in infection media, was added to each well. Assay plates were incubated for 4 days for HSV-1(HF) or 5 days for HSV-2 (G) at 37° C. with 5% CO₂. Following the 4- or 5-day incubation, the supernatant was replaced with 50 μL of CellTiterGlo 2.0 reagent (CTG, Promega, Cat #G9243), diluted 1:1 in PBS (Corning, Cat #21-040-CM). Relative light units (RLUs) were measured using a Tecan Spark plate reader. The EC₅₀ values were determined using GraphPad Prism 9 and Dotmatics.

### Example 2—COMPOUND 1 Resistance Selection

Clinical isolates HSV2-IS5, -IS18, -IS22, -IS25, -IS27, -IS28 and -IS30 were used for COMPOUND 1 resistance selections. Clinical isolates HSV2-IS25, -IS28, and -IS53 were used for acyclovir resistance selections. Vero cells were seeded in 6-well plates with 350,000 cells/well. After overnight culture, the cells were incubated with 0.1 μM or 0.2 μM of COMPOUND 1, or 10 μM or 20 μM of acyclovir (˜5× and 10×EC₅₀) for 1 hour. The cells were then infected with HSV-2 clinical isolates at a multiplicity of infection (MOI) of 0.05 in infection medium. After a 1-hour incubation, the virus inoculum was aspirated and then the cells were overlayed with maintenance medium containing 0.1 μM or 0.2 μM of COMPOUND 1, or 10 μM or 20 μM of acyclovir. The compound concentrations were increased by 2-fold when plaques were observed. Culture medium containing COMPOUND 1 or acyclovir was refreshed every 3 or 4 days until 100% CPE was observed. Supernatants were harvested and viruses were propagated in Vero cells with 0.1 μM COMPOUND 1, or 10 μM acyclovir. HSV genomic DNA was extracted from virus stocks using the QIAamp MinElute Virus Spin Kit (Qiagen, Cat #57704). For the COMPOUND 1 resistance selections, the known mutation region or full-length UL5 and UL52 genes were amplified from the extracted viral DNA. For the acyclovir resistance selections, the known mutation region or full-length UL23 and UL30 genes were amplified from the extracted viral DNA. Amplification and sequencing were performed using primers specific to UL5, UL52, UL23, and UL30. See, Collot, M., et al., 2016, and Wald, A., et al., 2014. Sanger sequencing results were analyzed by Sequencher (Gene Codes Corporation, USA) with the HSV-2 strain G complete genome (GenBank: OM370995.1) used as a reference sequence. Virus stocks from the resistance selections were titrated by plaque assay. Serial 10-fold dilutions of virus stocks were prepared and used to infect Vero cell monolayers in 6-well plates. After a 1-hour incubation, the virus inoculum was aspirated and then cells were overlayed with 2 mL of maintenance medium containing 1% carboxymethylcellulose (CMC, EMD Millipore, Cat #217277). Plates were then incubated for 5 days at 37° C. with 5% CO₂. Following the incubation, the supernatant was aspirated, and the cells were fixed with 4% formaldehyde (Sigma-Aldrich, Cat #252549). To visualize virus plaques the cells were stained with crystal violet solution (EMD Millipore, Cat #192-12).

Antiviral activity of COMPOUND 1 against resistance selection viruses was measured using a CPE reduction assay. Pre-seeded Vero cells were treated with 3-fold serially diluted compounds. All wells were normalized to a final concentration of 0.5% DMSO. Immediately following compound addition, cells were infected with resistance selection HSV-2 isolates at a multiplicity of infection (MOI) of 0.05 in infection medium and incubated for 5 days at 37° C. with 5% CO₂. Five days post-infection, the medium was replaced with 50 μL of diluted (1:1 ratio) CellTiter-Glo 2.0 reagent (CTG 2.0, Promega, Cat #G9243). The plates were immediately placed on a rotary shaker at 300 rpm for 10 minutes for signal stabilization. The relative light units (RLUs) were measured using a Tecan plate reader. EC₅₀ values were determined using GraphPad Prism 9 (GraphPad Software, USA).

### Example 3—Combination Studies

One day prior to infection, Vero cells were seeded in 96-well plates at a density of 10,000 cells/well for CompuSyn® analysis experiments and 8,000 cells/well for MacSynergy IITM analysis experiments. On the day of infection, compounds were added to the cells using the dilution scheme presented in Table 1 and 2. Immediately following compound addition, the cells were infected with HSV-1 or HSV-2 at a multiplicity of infection (MOI) of 0.05 or 0.1, respectively, in infection medium. In addition, DMSO treated wells were included to serve as either an HSV-1 or HSV-2 infection control (100% infection) or an uninfected control (0% infection). Five days post-infection, the medium was replaced with 50 μL of CellTiter-Glo 2.0 reagent (CTG 2.0, Promega, Cat #G9243) diluted 1:1 in PBS. The plates were immediately placed on a rotary shaker at 300 rpm for 10 minutes for signal stabilization. The relative light units (RLUs) were measured using a Tecan plate reader.

TABLE 1
Compounds Template for Combination Treatment using CompuSyn ® Analysis

| | | | concentration of compounds (nM) | | | | | | | |
|---|---|---|---|---|---|---|---|---|---|---|
| HSV-1 | Single | Acyclovir | 100,000 | 33,333 | 11,111 | 3,704 | 1,235 | 411.5 | 137.2 | 45.7 |
| | treatment | COMPOUND 1 | 1,000 | 333.3 | 111.1 | 37.04 | 12.35 | 4.115 | 1.372 | 0.457 |
| | Combo | Acyclovir | 100,000 | 50,000 | 20,000 | 10,000 | 5,000 | 2,000 | 500 | 100 |
| | treatment | COMPOUND 1 | 300 | 150 | 60 | 30 | 15 | 6 | 1.5 | 0.3 |
| HSV-2 | Single | Acyclovir | 50,000 | 16,667 | 5,556 | 1,852 | 617.3 | 205.8 | 68.6 | 22.9 |
| | treatment | COMPOUND 1 | 500 | 166.67 | 55.56 | 18.52 | 6.173 | 2.058 | 0.686 | 0.229 |
| | Combo | Acyclovir | 50,000 | 10,000 | 5,000 | 2,500 | 1,500 | 1,000 | 500 | 100 |
| | treatment | COMPOUND 1 | 500 | 100 | 50 | 25 | 15 | 10 | 5 | 1 |

TABLE 2
Plate Map for Combination Treatment using MacSynergy ™ Analysis

| Concentration of COMPOUND 1 (nM) | | | | | | | | | | | |
|---|---|---|---|---|---|---|---|---|---|---|---|
| 0 | 0.076 | 0.229 | 0.686 | 2.06 | 6.17 | 18.5 | 55.6 | 167 | 500 | DMSO | DMSO |

| | | | |
|---|---|---|---|
| Concentration | 50,000 | viral | uninfected |
| of acyclovir | 16,667 | infection | cell |
| (nM) | 5,556 | control | control |
| | 1,852 | | |
| | 617 | | |
| | 206 | | |
| | 68.6 | | |
| | 0 | | |

The Combination Indices (CI) for compound combinations were determined using CompuSyn® software. CompuSyn® uses the Loewe Additivity Model, which is based on the median-effect principal. See Chou, T. C. et al., (1984) and (2006). For this analysis, all drug combinations were carried out at a constant ratio. CI values were determined at ED₅₀, ED₇₅ and ED₉₀ inhibition levels. The degree of additivity, synergy or antagonism was ranked as indicated in Table 3.

TABLE 3
CompuSyn ® Analysis Interpretation

| CI value | Description |
|---|---|
| <0.1 | Very Strong Synergism |
| 0.1-0.3 | Strong Synergism |
| 0.3-0.7 | Synergism |
| 0.7-0.85 | Moderate Synergism |
| 0.85-0.90 | Slight Synergism |
| 0.90-1.10 | Nearly Additive |
| 1.10-1.20 | Slight Antagonism |
| 1.20-1.45 | Moderate Antagonism |
| 1.45-3.3 | Antagonism |
| 3.3-10 | Strong Antagonism |
| >10 | Very Strong Antagonism |

The synergism/antagonism volumes for the drug combinations used were calculated using MacSynergy IITM software. MacSynergy IITM uses Bliss Independence to calculate an additive value (based on single drug alone) for each drug-drug combination point. Theoretical additive values are compared to experimental data to determine whether a drug-drug combination is synergistic (if theoretical<experimental) or antagonistic (if theoretical>experimental). When plotted on a 3D graph, the volume of data representing synergism and antagonism across all drug concentrations is measured. Volume (in μM²%) is statistically evaluated using a 95% confidence level and used to predict the degree of synergy according to the scale shown in Table 4.

TABLE 4
MacSynergy II ™ Analysis Interpretation

| | |
|---|---|
| Synergy Volume (log volume) | Result |

| | | |
|---|---|---|
| <25 μM²%, | (<2) | Insignificant synergy (additive) |
| 25-50 μM²%, | (2-5) | Minor but significant synergy |
| 50-100 μM²%, | (5-9) | Moderate synergy |
| >100 μM²%, | (>9) | Strong synergy |

### Example 4—Virus Specificity Assays

VZV, EBV, HHV-6, and BK antiviral assays were performed at WuXi. HCMV, RSV, HCV replicon, HBV, and HDV antiviral assays were performed at ASMB. A summary of the details for each antiviral assay is presented in Table 5. COMPOUND 1 and control compound concentrations for each assay are listed in Table 6.

TABLE 5
List of antiviral assays, experimental details, and site at which the assay was conducted.

| Virus | Cell Type | | | | | Site of |
|---|---|---|---|---|---|---|
| (strain) | (quantity/well) | Control | MOI | Assay Type | Assay Readout | Assay |
| VZV (Oka) | MRC-5 (20,000) | ACV | 0.05 | qPCR^{§} | CCK8 detection | WuXi |
| EBV (B95) | B95-8 (20,000) | ACV | n.a.* | qPCR^{§} | EBNA1 detection | WuXi |
| HHV-6B (SF) | PBMC (40,000) | GCV | n.a.* | qPCR^{§} | U22 detection | WuXi |
| HCMV (AD169) | MCR-5 (5,000) | LTV | 1:400** | CPE^{#} | WST-1 | ASMB |
| BK (MM) | RPTEC/TERT1 (10,000) | CFV | n.a.* | IFA^{†} | SV-40 T-antigen | WuXi |
| RSV (A2) | HEp-2 (1,500) | RLV | 1:300** | CPE^{#} | Luciferase/CTG | ASMB |
| HCV (1b) | HCV-1b replicon Huh7 | SOF | n.a.* | HCV | NanoGlo | ASMB |
| | (7,000) | | | replicon | Luciferase | |
| HBV (AD38) | HepG2-NTCP (50,000) | MyrB | 50 | ELISA^{‡} | HBeAg | ASMB |
| HDV (3D) | HepG2-NTCP (10,000) | MyrB | 1:2** | In-cell | HDAg | ASMB |
| | | | | ELISA^{‡} | | |

*n.a., not applicable;
**Volumetric ratio of virus stock for infection;
^{§}qPRC, quantitative real-time PCR;
^{#}CPE, cytopathic effect;
^{†}IFA, immunofluorescence assay;
^{‡}ELISA, enzyme-linked immunosorbent assay

TABLE 6
Dilution series for COMPOUND 1 and control compounds.

| | COMPOUND | COMPOUND | | | | | | | |
|---|---|---|---|---|---|---|---|---|---|
| Condition | 1^{§§} | 1^{##} | ACV | GCV | LTV | CFV | RLV | SOF | MyrB |
| 1 | 20 | 20 | 100 | 100 | 0.5 | 1.5 | 0.1 | 10 | 1.0 |
| 2 | 5 | 6.67 | 33.3 | 33.3 | 0.125 | 0.5 | 0.025 | 3.33 | 0.25 |
| 3 | 1.25 | 2.22 | 11.1 | 11.1 | 0.313 | 0.167 | 0.006 | 1.11 | 0.063 |
| 4 | 0.313 | 0.741 | 3.7 | 3.7 | 0.008 | 0.056 | 0.002 | 0.37 | 0.016 |
| 5 | 0.078 | 0.247 | 1.23 | 1.23 | 0.002 | 0.019 | 3.90 × 10⁻⁴ | 0.124 | 0.004 |
| 6 | 0.019 | 0.082 | 0.412 | 0.412 | 4.88 × 10⁻⁴ | 0.006 | 9.77 × 10⁵ | 0.041 | 0.001 |
| 7 | 0.004 | 0.027 | 0.137 | 0.137 | 1.22 × 10⁻⁴ | 0.002 | 2.44 × 10⁻⁵ | 0.014 | 2.0 × 10⁻⁴ |
| 8 | 0.001 | 0.009 | 0.046 | 0.046 | 3.05 × 10⁻⁵ | 6.90 × 10⁻⁴ | 6.10 × 10⁻⁶ | 0.005 | 1.0 × 10⁻⁴ |

^{§§}Dilution series for HCMV, RSV, HBV, and HDV antiviral assays.
^{##}Dilution series for VZV, EBV, HHV-6, BK, and HCV antiviral assays.

### CMV Assay

MRC-5 cells were grown to 80-90% confluency, washed twice with DPBS (Thermo Fisher, Cat #14190-235), and detached from the culture flask using 0.25% Trypsin-EDTA (Gibco, Cat #25200-056) at 37° C. for 3 minutes. Detached cells were spun at 500 g for 5 min and the cell pellet was resuspended in growth media. MRC-5 cells were seeded onto 96-well flat clear tissue culture treated microplates (Corning, Cat #3596) in growth media at the cell density noted in Table 5. The following day, growth media was replaced with 50 μL of infection media containing either COMPOUND 1 or LTV using the dilution scheme presented in Table 6. All wells were normalized to a final DMSO concentration of 0.5%, 50 μL of HCMV (AD169), diluted 400-fold in infection media, was added to each well followed by 7-day incubation at 37° C. with 5% CO₂. After the 7-day incubation, 10 μL of tetrazolium salt WST-1 (CellBio Labs, Cat #CBA253) was added to each well, followed by a 4-hour incubation at 37° C. After the incubation with WST-1, cell viability was determined by measuring the absorbance of formazan formation at 440 nM. Assay plates were read using a Tecan Spark plate reader. The EC₅₀ values were determined using GraphPad Prism 9 and Dotmatics.

### RSV Assay

HEp-2 cells were grown to 70-90% confluency, washed once with DPBS, and detached from the culture flask using 0.25% Trypsin-EDTA at 37° C. for 3 minutes. Detached cells were spun at 500 g for 5 min and the cell pellet was resuspended in growth media. HEp-2 cells were seeded onto 96-well flat clear bottom black polystyrene tissue culture-treated microplates (Corning, Cat #3904) in growth media at the cell density noted in Table 5. The following day, growth media was replaced with 50 μL of infection media containing either COMPOUND 1 or RLV using the dilution scheme presented in Table 6. All wells were normalized to a final DMSO concentration of 0.5%, 50 μL of RSV (A2), diluted 300-fold in infection media, was added to each well and then incubated for 5 days at 37° C. with 5% CO₂. Following the 5-day incubation, the supernatant was replaced with 50 μL of CellTiterGlo 2.0 reagent (CTG, Promega, Cat #G9243) diluted 1:1 in phosphate buffered saline (Corning, Cat #21-040-CM). Relative light units (RLUs) were measured using a Tecan Spark plate reader. The EC₅₀ values were determined using GraphPad Prism 9 and Dotmatics.

### HCV Assay

HCV-1b replicon Huh7 cells were grown to 70-90% confluency, washed once with DPBS, and detached from the culture flask using 0.25% Trypsin-EDTA at 37° C. for 3-4 minutes. Detached cells were spun at 500 g for 5 min and the cell pellet was resuspended in infection media. HCV-1b replicon Huh7 cells were seeded into 96-well half area clear bottom plates (Greiner, Cat #650201) in infection media at the cell density noted in Table 5. COMPOUND 1 or SOF was dispensed into designated wells using the dilution scheme presented in Table 6 and incubated for 2 days at 37° C. with 5% CO₂. All wells were normalized to a final DMSO concentration of 0.5%. Following the incubation, Nano-Glo Luciferase reagent (Promega, Cat #N1120) was prepared according to manufacturer instructions and 25 μL of prepared reagent was added to each well. Assay plates were placed on orbital shaker for mixing ˜3 min and then RLUs were measured using a Tecan Spark plate reader. The EC₅₀ values were determined using GraphPad Prism 9 and Dotmatics.

### HBV Assay

HepG2-NTCP cells were grown to 80% confluency, washed once with DPBS, and detached from the culture flask using 0.25% Trypsin-EDTA at 37° C. for 5 minutes. Detached cells were spun at 500 g for 5 min and the cell pellet was resuspended in infection media. HepG2-NTCP cells were seeded into 96-well white opaque bottom plates (Greiner, Cat #655074) in infection media at the cell density noted in Table 5. COMPOUND 1 or MyrB was dispensed into designated wells using the dilution scheme presented in Table 6. All wells were normalized to a final DMSO concentration of 1.0%. The HepG2-NTCP cells were then infected with 50 μL HBV (AD38) at an MOI of 50 and incubated for 24 hours at 37° C. with 5% CO₂. Following incubation, infection media was replaced with 100 μL DMEM+5% FBS+1× Pen-Strep and incubated for another 4 days at 37° C. with 5% CO₂.

After the 4-day incubation, supernatant was harvested by pelleting cells in assay plates at 500 g for 5 minutes. 10 μL of supernatant was transferred into a 96-well white opaque bottom plate previously coated with 62.5 ng mouse monoclonal anti-HBV HBe antigen (Biocheck, Cat #70426 Lot #RN-31725) as the capture antibody. Secondary anti-HBeAg-Horse radish peroxidase (HRP) conjugated monoclonal antibody (Sigma, Cat #A7030-100 g) was diluted 1:10,000 in 1×PBS (Thermo, Cat #J61196.AP) containing 0.1% Tween-20 (PBS-T; Thermo, Cat #AAJ20605AP), resulting in a total volume of 100 μL per well. ELISA plates containing secondary antibody and assay supernatant were incubated at room temperature for 2 hours with shaking. After 2 hours, the assay plates are washed six times with 1×PBS-T, followed by the addition of 50 μL of pre-diluted 1:1 chemiluminescent HRP substrate (EMD Millipore, Cat #WBKLS0500). Plates were incubated for 1 minute at room temperature, in the dark, to stabilize the chemiluminescent reaction, followed by measuring luminescence on a Tecan Spark plate reader. The EC₅₀ values were determined using GraphPad Prism 9 and Dotmatics HDV Assay

In preparation for compound dispensing, 50 μL of 2×HDV was added to each well of collagen-coated 96-well clear bottom plates (Corning, Cat #354649). COMPOUND 1 or MyrB was dispensed into designated wells using the dilution scheme presented in Table 6. All wells were normalized to a final DMSO concentration of 1.0%. HepG2-NTCP cells were grown to 50-90% confluency, washed once with DPBS, and detached from the culture flask using 0.25% Trypsin-EDTA at 37° C. for 5 minutes. Detached cells were spun at 500 g for 5 min and the cell pellet was resuspended in infection media. 50 μL of HepG2-NTCP cells were seeded into the 96-well plates containing 2×virus at a density of 1×10⁴ cells per well (Table 4). After a 24-hour incubation, infection media was replaced with media supplemented with 250 nM Lonafarnib (Selleckchem, Cat #SCH66336) and incubated for an additional 5 days at 37° C. with 5% CO₂. After the 4-day incubation, the assay plates were washed once with PBS, then cells were fixed with 4% paraformaldehyde (EMS Cat #15710-S) in PBS for 30 minutes at room temperature. After fixing step, cells were permeabilized with PBS-T for 15 minutes at room temperature. Following fixation, assay plates were washed three times with PBS and then blocked in LICOR intercept buffer (Sigma Cat #648463-50ML) for 45 minutes at room temperature. 100 μL per well of primary antibody (Kerafast mouse monoclonal clone FD3A7 antibody Cat #EHD001) diluted in 1:100 LICOR intercept buffer containing 0.05% Tween-20 was added to all wells followed by an 18-hour incubation at 4° C. and then three 5-minute PBS-T washes. A 1:800 dilution of LICOR goat anti-mouse IRDye800CW secondary antibody (Sigma Cat #926-32210) and 1:500 dilution of LICOR CellTag 700 stain (Sigma, Cat #926-41090) were added to assay plates at a volume of 100 μL per well and the plates were incubated at room temperature for 1 hour in the dark. The plates were then washed three times with PBS. One final 5-minute wash was performed with 100 μL PBS per well. PBS was aspirated and plates were imaged on a LICOR Odyssey scanner. The EC₅₀ values were determined using GraphPad Prism 9 and Dotmatics.

### Example 5—Cytotoxicity Assays

Huh7, HEK-293, HepG2, HeLa-H1A, MOLT-4, and NCI-H226 cells were washed once with PBS, detached using 0.25% Trypsin-EDTA (Gibco, Cat #25200-056) and then plated in 96-well flat clear bottom black polystyrene tissue culture-treated microplates (Corning, Cat #3904) at a cell density of 5,000 cells/well (MOLT-4 and NCI-H226) or 10,000 cells/well (Huh7, HEK-293, HepG2, and HeLa-H1A) in 90 μL of the appropriate cell culture media (see Section 3.1.2). The cells were then treated with COMPOUND 1 or puromycin using 1:3-fold serial dilutions at a starting concentration of 30 μM. After compound addition, the cells were incubated at 37° C. for 96 hours. Following the incubation, 50 μL of CellTiterGlo 2.0 reagent (CTG 2.0, Promega, Cat #G9243), diluted 1:1 in phosphate buffered saline (Corning, Cat #21-040-CM), was added to each well. The plates were then placed on a rotary shaker at 250 rpm for 2 minutes followed by a 10-minute benchtop incubation to stabilize the signal. Relative light units (RLUs) were measured using a Tecan Spark plate reader. The CC₅₀ values were determined using GraphPad Prism 9 and Dotmatics.

PBMCs were stimulated by gently resuspending the cells in fresh RPMI growth media supplemented with 10 U/mL recombinant interleukin-2 (rIL-2, Sigma, Cat #I2644-10UG) and 1% phytohemagglutinin M (PHA-M, ThermoFisher, Cat #10576015) at 37° C. for 72 hours. Following stimulation, the PBMCs were then centrifuged at 100 g for 8 minutes and plated in 96-well flat bottom opaque white plates at a density of 100,000 cells/well in 180 μL of RPMI growth media. The cells were then treated with COMPOUND 1 or puromycin using 1:3-fold serial dilutions at a starting concentration of 30 μM. After compound addition, the cells were incubated at 37° C. for 96 hours. Following the incubation, 50 μL of CTG 2.0 reagent was added to wells and the plates were placed on a rotary shaker at 250 rpm for 2 minutes, followed by a 10-minute benchtop incubation to stabilize the signal. RLUs were measured using a Tecan Spark plate reader. The CC₅₀ values were determined using GraphPad Prism 9 and Dotmatics.

MT-4 cells were centrifuged at 500 g for 5 minutes and resuspended in RPMI growth medium. Following resuspension, the cells were then plated in 96-well flat bottom opaque white plates at a density of 10,000 cells/well in 100 μL of RPMI growth media. The cells were then treated with COMPOUND 1, puromycin, NSC617145, MSC19630, or ML216 using 1:3-fold serial dilutions at a starting concentration of 30 μM. After compound addition, the cells were incubated at 37° C. for 96 hours. Following the incubation, 100 μL of CTG 2.0 reagent was added to wells and the plates were placed on a rotary shaker at 250 rpm for 2 minutes, followed by a 10-minute benchtop incubation to stabilize the signal. RLUs were measured using a Tecan Spark plate reader. The CC₅₀ values were determined using GraphPad Prism 9 and Dotmatics.

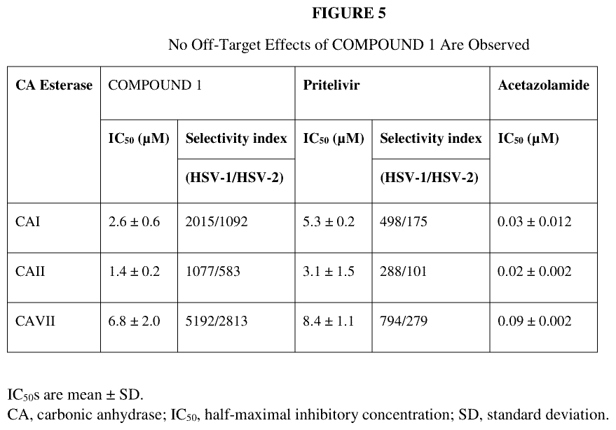

FIG. 5 describes that no off-target effects of Compound 1 are observed in vitro. Compound 1 inhibits HSV over potential human off-target CAs I, II, and III with a selectivity index of >500. A favorable safety profile of Compound 1 is observed in rats and dogs in 28-day oral toxicity studies, with high safety margins relative to the predicted human equivalent dose (data not shown).

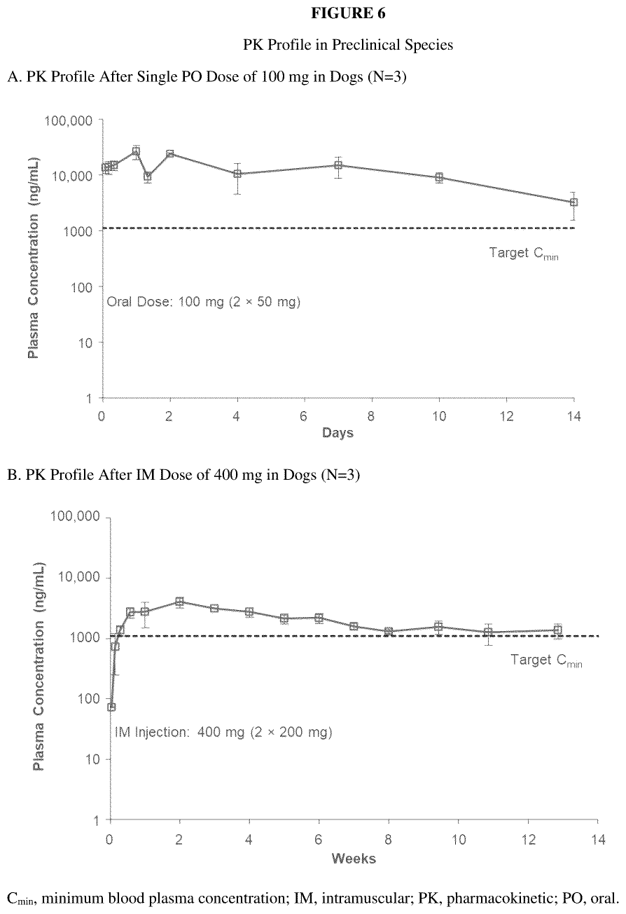

FIG. 6 describes pK profile of Compound 1 after a single dose of 100 mg in dogs (FIG. 6A) and pK profile of Compound 1 after IM dose of 400 mg in dogs (FIG. 6B). In dog PK studies, an oral or injectable dose of Compound 1 results in sustained therapeutic plasma concentrations for approximately 2 weeks and more than 3 months, respectively, demonstrating the long-acting potential of Compound 1.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The examples described herein are provided by way of illustration only and not by way of limitation, Those skilled in the art will readily recognize a variety of parameters that could be changed or modified to yield essentially similar results.

1. A method for the treatment of an HSV infection in a subject in need thereof, the method comprising: administering to the subject a therapeutically effective amount of Compound 1: 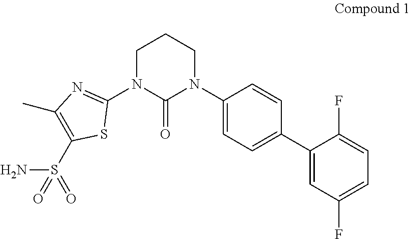 [SMILES: Cc1nc(N2CCCN(c3ccc(-c4cc(F)ccc4F)cc3)C2=O)sc1S(N)(=O)=O]
or a pharmaceutically acceptable salt thereof; wherein the virus is unresponsive to treatment with one or more first antiviral therapy.

2. The method of claim 1, wherein the first antiviral therapy is a nucleoside analogue or a helicase-primase inhibitor.

3. The method of claim 2, wherein the first antiviral therapy is a nucleoside analogue, the nucleoside analogue is acyclovir.

4. The method of claim 2, wherein the first antiviral therapy is a helicase-primase inhibitor, the helicase-primase inhibitor is pritelivir.

5. The method of claim 1, wherein the infection is a Herpes Simplex Virus (HSV) infection.

6. The method of claim 5, wherein the HSV is an HSV-1 or HSV-2.

7. The method of claim 5, wherein the HSV comprises one or more mutations.

8. The method of any one of claims 5-7, wherein the HSV comprises a T288M mutation in the UL23 gene.

9. The method of any one of claims 1-8, wherein the infection is resistant to treatment with one or more antiviral therapy.

10. The method of any one of claims 1-8, wherein Compound 1 is about 400 fold more potent than acyclovir.

11. The method of any one of claims 1-8, wherein Compound 1 is about 4 fold more potent than pritelivir.

12. A method for the treatment of a viral infection unresponsive to treatment with one or more first antiviral therapies in a subject in need thereof, the method comprising: administering to the subject a therapeutically effective amount of a pharmaceutical composition comprising Compound 1: 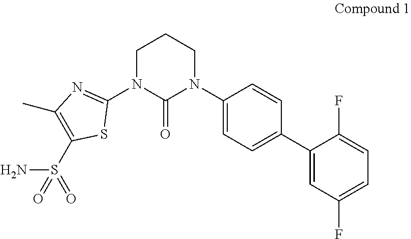 [SMILES: Cc1nc(N2CCCN(c3ccc(-c4cc(F)ccc4F)cc3)C2=O)sc1S(N)(=O)=O]
or a pharmaceutically acceptable salt thereof and a pharmaceutically acceptable excipient.

13. The method of claim 11, wherein the first antiviral therapy is a nucleoside analogue or a helicase-primase inhibitor.

14. The method of claim 12, wherein the nucleoside analogue is acyclovir.

15. The method of claim 12, wherein the helicase-primase inhibitor is pritelivir.

16. The method of claim 11, wherein the infection is an HSV infection.

17. The method of claim 15, wherein the HSV is an HSV-1 or HSV-2.

18. The method of claim 11, wherein the HSV comprises one or more mutations.

19. The method of any one of claims 16-18, wherein the HSV comprises a T288M mutation in the UL23 gene.

20. The method of any one of claims 11-18, wherein the infection is resistant to treatment with one or more antiviral therapies.

21. The method of any one of claims 11-18, wherein Compound 1 is about 400 fold more potent than acyclovir.

22. The method of any one of claims 11-18, wherein Compound 1 is about 4 fold more potent than pritelivir.

23. A method for the treatment of HSV infection previously treated with acyclovir, in a subject in need thereof, the method comprising: administering to the subject a therapeutically effective amount of Compound 1: 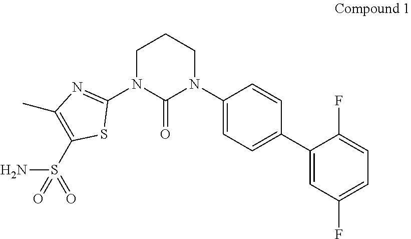 [SMILES: Cc1nc(N2CCCN(c3ccc(-c4cc(F)ccc4F)cc3)C2=O)sc1S(N)(=O)=O]
or a pharmaceutically acceptable salt thereof.

24. A method for the treatment of HSV infection previously treated with pritelivir, in a subject in need thereof, the method comprising: administering to the subject a therapeutically effective amount of Compound 1: 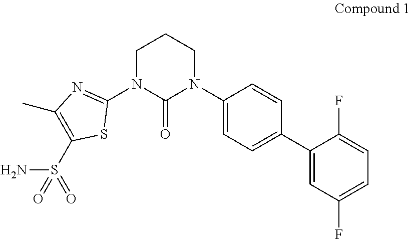 [SMILES: Cc1nc(N2CCCN(c3ccc(-c4cc(F)ccc4F)cc3)C2=O)sc1S(N)(=O)=O]
or a pharmaceutically acceptable salt thereof.

25. A method of treatment of an HSV infection previously treated with an antiviral therapy in a subject in need thereof, the method comprising: administering to the subject a therapeutically effective amount of Compound 1: 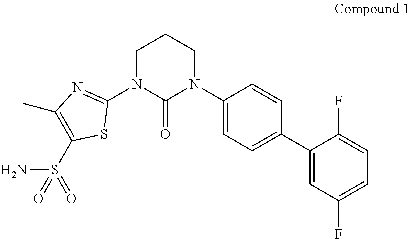 [SMILES: Cc1nc(N2CCCN(c3ccc(-c4cc(F)ccc4F)cc3)C2=O)sc1S(N)(=O)=O]
or a pharmaceutically acceptable salt thereof;
wherein the HSV virus comprises one or more mutations.

26. The method of claim 25, wherein the HSV virus comprises a T288M mutation in the UL23 gene.
